(19) United States
(12) Patent Application Publication
(10) Pub. No.: US 2026/0240863 A1
(43) Pub. Date: Aug. 20, 2026

(54) COMBINATION OF ROGINOLISIB AND HDAC INHIBITOR IN THE TREATMENT OF HAEMATOLOGICAL MALIGNANCY

(71) Applicant: IONCTURA SA, Genève (CH)

(72) Inventors: Karolina NIEWOLA-STASZKOWSKA, Genève (CH); Michael LAHN, Genève (CH); Giusy DI CONZA, Genève (CH); Francesco BERTONI, Bellinzona (CH); Chiara TARANTELLI, Bellinzona (CH)

(21) Appl. No.: 19/161,841

(22) PCT Filed: Mar. 1, 2024
(86) PCT No.: PCT/EP2024/055475

§ 371 Date: Sep. 3, 2025

(30) Foreign Application Priority Data
Mar. 3, 2023 (GB) 2303191.7
May 31, 2023 (GB) 2308112.8

(51) Int. Cl. A61K 31/5377; A61K 31/167; A61K 31/18; A61K 38/15; A61P 35/00

(52) U.S. Cl. CPC A61K 31/5377; A61K 31/167; A61K 31/18; A61K 38/15; A61P 35/00

(57) ABSTRACT

A PI3K-delta inhibitor or a pharmaceutically acceptable salt thereof, for use in the treatment of a haematological malignancy in a subject, wherein said treatment comprises the separate, sequential or simultaneous administration of i) said PI3K-delta inhibitor, or a pharmaceutically acceptable salt thereof, and ii) a HDAC inhibitor, or a pharmaceutically acceptable salt thereof, to said subject. The treatment can also further comprise the separate, sequential or simultaneous administration of iii) a further chemotherapeutic agent, to said subject.

The present disclosure relates to methods of treating haematological malignancies in a patient in need thereof.

The application claims the benefit of priority to GB application No. 2303191.7, filed on 3 Mar. 2023, and GB application No. 2308112.8, filed on 31 May 2023, which are incorporated herein by reference in its entirety.

### BACKGROUND

Haematological malignancies, also so known as blood cancers, are malignant neoplasms derived mostly from the myeloid and lymphoid cell lineages. Lymphoma, lymphocytic leukaemia, and myeloma are derived from the lymphoid lineage, while acute and chronic myelogenous leukemia, myelodysplastic syndromes and myeloproliferative diseases are of myeloid origin.

Haematological malignancies are a significant cause of morbidity and mortality worldwide, with almost 1,000,000 new cases and over 600,000 deaths annually (Jephcote 2020). Several agents contribute to the development of blood cancers, including occupational, lifestyle, and hereditary risk factors. Haematological malignancies are a heterogeneous disease for which outcomes can vary. Indolent lymphoma and chronic leukaemia, such as follicular lymphoma (FL), marginal zone lymphoma (MZL), chronic lymphocytic leukaemia (CLL) and small lymphocytic lymphoma (SLL), remain incurable chronic diseases, requiring patients to undergo repeated exposures to toxic therapies. For aggressive lymphoma and acute leukaemia, modern treatment regimens result in long-term survival rates ranging from >80% for Hodgkin lymphoma to ~60-65% for diffuse large B-cell lymphoma (DLBCL) and acute lymphoblastic leukaemia (ALL), and to <30% for peripheral T-cell lymphoma (PTCL) and acute myeloid leukaemia (AML) (Intlekofer and Younes 2014, Kantarjian 2021). More effective therapies are needed to improve outcome for patients with haematologic malignancies. Furthermore, even for those patients who are cured by combination chemotherapy, often used in conjunction with radiation, this therapy can result in long-term toxicities that impair health, predispose patients to secondary malignancies, and negatively impact quality of life.

Targeted therapies including phosphatidylinositol 3-kinase (PI3K) inhibitors and histone deacetylase (HDAC) inhibitors have changed the treatment landscape for patients with haematological malignancies. PI3Ks play a central role in the regulation of key intercellular pathways including growth, proliferation, survival, migration and differentiation, and dysregulation of the PI3K pathway is one of the most frequent pathogenic events in cancer (Kienle and Stilgenbauer, 2021). HDAC modulates cell proliferation and angiogenesis and plays an essential role in cell growth, and up-regulated HDACs are present in many cancer types (Chen 2020). Thus PI3K and HDAC are rational therapeutic targets in haematological malignancies.

Although PI3K and HDAC inhibitors have demonstrated impressive clinical outcomes across a variety of haematological malignancies, certain subtypes are characterized by significantly higher response rates than others and resistance develops to these inhibitors eventually leading to clinical disease progression. Also both types of inhibitors come with severe adverse effects limiting their therapeutic applicability, and in the case of PI3K inhibitors even resulting in drug withdrawals from the market (Chen 2020, Richardson 2022).

While much progress has been made in the treatment of haematological malignancies, there is increasing demand for targeted therapy for haematological malignancies. Many patients who have such cancers live with an incurable disease. Accordingly, it is important to continue to find new treatments for patients with incurable cancer that are more effective.

### SUMMARY

The present invention is directed to a new treatment for patients with haematological malignancies. In some embodiments, disclosed is a method of treating a haematological malignancy in a subject in need thereof, comprising administering to the subject a first amount of a compound of Formula I:

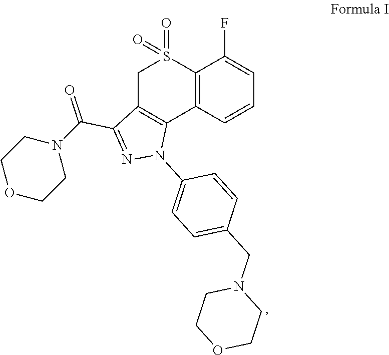 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and a second amount of a HDAC inhibitor or a pharmaceutically acceptable salt thereof. In the method, the first amount and the second amount together comprise a therapeutically effective amount. The compound of Formula I may be referred to herein as “Compound 1”. In some of these embodiments, a third amount of a further chemotherapeutic agent is administered.

In some embodiments, disclosed is Compound 1, or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a HDAC inhibitor, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is a HDAC inhibitor, or a pharmaceutically acceptable salt thereof, for use in the treatment of a haematological malignancy in a subject, wherein said treatment comprises the separate, sequential or simultaneous administration of i) said a HDAC inhibitor, or a pharmaceutically acceptable salt thereof, and ii) Compound 1, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is the use of Compound 1, or a pharmaceutically acceptable salt thereof, in the manufacture of a medicament for use in the treatment of a haematological malignancy, wherein said treatment comprises the separate, sequential or simultaneous administration of i) said medicament comprising Compound 1, or a pharmaceutically acceptable salt thereof, and ii) a HDAC inhibitor, or a pharmaceutically acceptable salt thereof, to said subject. In some of these embodiments, the treatment further comprises the separate, sequential or simultaneous administration of iii) a further chemotherapeutic agent, to said subject.

In the above embodiments, the haematological malignancy may be lymphoma, leukaemia, myeloma, myelodysplastic syndrome and myeloproliferative disease.

In some embodiments, disclosed is a pharmaceutical product comprising i) Compound 1 or a pharmaceutically acceptable salt thereof, and ii) a HDAC inhibitor or a pharmaceutically acceptable salt thereof. In some of these embodiments, the pharmaceutical product further comprises iii) a further chemotherapeutic agent.

In some embodiments, disclosed is a kit comprising: a first pharmaceutical composition comprising Compound 1, or a pharmaceutically acceptable salt thereof; a second pharmaceutical composition comprising a HDAC inhibitor, or a pharmaceutically acceptable salt thereof; and instructions for using the first and second pharmaceutical compositions in combination. In some of these embodiments, the kit further comprises a third pharmaceutical composition comprising a further chemotherapeutic agent, and the instructions are for using the first, second and third pharmaceutical compositions in combination.

The combination of Compound 1 and a HDAC inhibitor (as well as the further chemotherapeutic agent, if present) may be synergistic, and/or may otherwise result in improved treatment outcomes or patient prognosis, for example the reduction of side effects and improved tolerability, when compared to current monotherapies or combination therapies. This may result from Compound 1 blocking survival pathways that are activated once HDAC inhibitors are given and vice versa.

### BRIEF DESCRIPTION OF THE DRAWINGS

FIG. 1 shows the Dose response data of the combination of Compound 1 and Vorinostat in the HH cell line, a cutaneous T-cell lymphoma cell line.

FIG. 2 shows Dose response data of the combination of Compound 1 and Romidepsin in the HH cell line, a cutaneous T-cell lymphoma cell line.

### DETAILED DESCRIPTION

Compound 1 is example 339 in WO2011/058149, which document is incorporated herein by reference in its entirety. Its structure is according to Formula I:

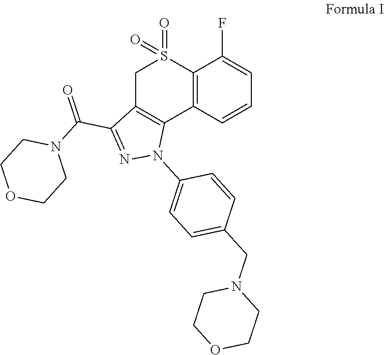 [SMILES: O=C(c1nn(-c2ccc(CN3CCOCC3)cc2)c2c1CS(=O)(=O)c1c(F)cccc1-2)N1CCOCC1]

In IUPAC nomenclature, the above Compound 1 may be referred to as 6-Fluoro-3-(morpholin-4-ylcarbonyl)-1-[4-(morpholin-4-ylmethyl)phenyl]-1,4-dihydrothiochromeno[4,3-c]pyrazole 5,5-dioxide. Alternatively, the structural formula shown above may be described as [6-fluoro-1-(4-morpholin-4-yl-methylphenyl)-5,5-dioxo-4,5-dihydro-1H-5λ6-thiochromeno[4,3-C]pyrazol-3-yl]-morpholin-4-yl-methanone.

Compound 1 can be prepared and characterized as described in published patent application WO 2011/058149 A1 (see compound 339 on p. 69; the preparation on p. 303-307; and the characterization on p. 481 with p. 414-418), which information is specifically incorporated herein by reference.

Based on the process disclosed in WO 2011/058149 A1, the authors of Haselmayer 2014 describe a five-step preparation procedure for the compound. This procedure starts with reaction of 8-fluoro-2,3-dihydro-4Hthiochromen-4-one with diethyl oxalate in the presence of sodium ethoxide. The intermediate is cyclized with 4-(4-hydrazinylbenzyl) morpholine to form a pyrazole ring. The thioether is then oxidized to the corresponding sulfone by reaction with meta-chloroperbenzoic acid, followed by saponification of the ethyl ester into the corresponding acid and subsequent coupling with morpholine to yield the compound of formula I.

Alternatively, the intermediate of the reaction of 8-fluoro-2,3-dihydro-4H-thiochromen-4-one with diethyl oxalate in the presence of sodium ethoxide is cyclized with 4-hydrazinobenzoic acid. The benzoic acid is reduced using borane-THE complex and the thioether is oxidized to the corresponding sulfone by reaction with meta-chloroperbenzoic acid. Saponification of the ethyl ester into the corresponding acid and chlorination of both the acid and alcohol with excess thionyl chloride in the presence of dimethylformamide and subsequent coupling with morpholine then yields Compound 1.

Compound 1 may be provided as a pharmaceutically acceptable salt. Suitable pharmaceutically acceptable salts are known in the art. Some pharmaceutically acceptable salts of Compound 1 are described in WO2014/121901, which is incorporated by reference in its entirety.

As used herein, Compound 1 is provided as an anhydrous hemifumarate salt (formula illustrated). Its synthesis and characterisation are described in WO2014/121901 (page 4). It is referred to as solid form A1. A hemifumarate hydrate (H1) has also been identified. The anhydrous hemifumarate salt used is crystalline and has a powder X-ray peak list as described in WO2014/121901. It will be appreciated that the findings of the invention are not limited to use of this solid form, although it is preferred.

Accordingly, in some cases Compound 1 is administered as the hemifumarate salt (Formula Ia). However, it will be understood that the invention is not so limited, and other solid forms (for example, other pharmaceutically acceptable salts) are envisaged.

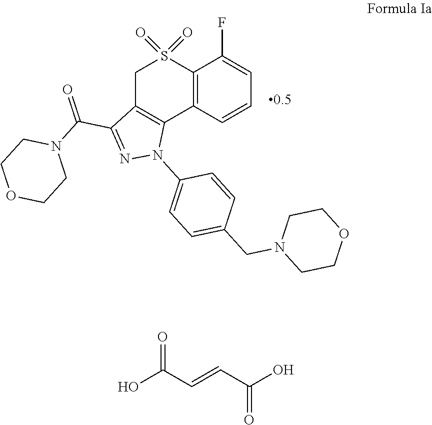 [SMILES: O=C(c1nn(-c2ccc(CN3CCOCC3)cc2)c2c1CS(=O)(=O)c1c(F)cccc1-2)N1CCOCC1 ; O=C(O)/C=C/C(=O)O]

Haselmayer 2014 et al. also describe the characterisation of the compound as highly selective PI3Kδ inhibitor. Tarantelli 2022 further describes the activity of Compound 1 in lymphoma cell lines and Carlo-Stella 2022 reports the data on the first dose cohort of the clinical study of Compound 1 in FL patients (NCT04328844).

Histone deacetylases (HDACs) are enzymes that play a key role in the epigenetic regulation of gene expression by remodeling chromatin through removal of the acetyl group from histones (Shanmugam 2022). In humans 18 HDACs have been identified to date and based on their homology to yeast HDACs, they are categorized into different classes. The term HDAC inhibitor includes targeted and selective inhibitors to one of the types of HDACs or targeted inhibitors to more than one or all the types of HDACs. HDAC inhibitors include but are not limited to Abexinostat (PCI-24781), Pracinostat (SB939), Quisinostat (JNJ-26481585), Tefinostat (CHR-2845), Panobinostat (LBH589), Belinostat (PXD101), Givinostat (ITF2357), Tucidinostat (CS-055, HBI-8000), Vorinostat (suberoylanilide hydroxamic acid, SAHA), Mocetinostat (MGCD0103), Valproic acid (VAL), Entinostat (MS275), Romidepsin (depsipeptide, FK228), and Trapoxin (TPX).

Vorinostat (also known as Suberoylanilide hydroxamic acid-SAHA) is an orally bioavailable HDAC inhibitor, approved by the FDA in 2006 for treatment of cutaneous T-cell lymphoma (CTCL) (Bondarev 2021). Clinically Vorinostat is evaluated in patients with several haematological malignancies including multiple myeloma, AML, ALL, myelodysplastic syndromes, DBCL (Diffuse large B-cell lymphoma), CLL, SLL, leukemia and lymphoma. The most common toxic effects observed for Vorinostat include thrombocytopenia, anemia, diarrhea, fatigue, nausea, anorexia, weight decrease and dysgeusia. Thromboembolic events, particularly pulmonary embolism (4%), were observed as the most common life-threatening events.

Vorinostat has the following structure:

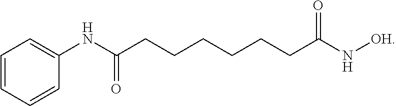 [SMILES: O=C(CCCCCCC(=O)Nc1ccccc1)NO]

The IUPAC name of Vorinostat is N-Hydroxy-N′-phenyloctanediamide. Its synthesis and characterisation are described in US2008/0194692 A1.

Belinostat is an intravenously administered HDAC inhibitor approved by the FDA for peripheral T-cell lymphoma (PTCL) in 2014 (Bondarev 2021). Belinostat is investigated in patients with several haematological malignancies including non-Hodgkin lymphoma, DBCL, MCL (mantle cell lymphoma), PTCL, lymphoma, large cell lymphoma, myelodysplastic syndromes, AML, ALL (Acute lymphocytic leukemia), CML (Chronic myelogenous leukemia), and MM (multiple myeloma). The most common non-heamatological toxicities observed for belinostat are nausea, fatigue, pyrexia, anemia, and vomiting.

Belinostat has the following structure:

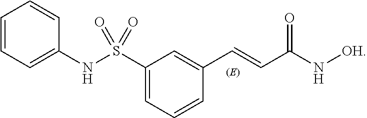 [SMILES: O=C(/C=C/c1cccc(S(=O)(=O)Nc2ccccc2)c1)NO]

The IUPAC name of Beliniostat is (2E)-N-Hydroxy-3-[3-(phenylsulfamoyl)phenyl]prop-2-enamide. Belinostat is sold under the trade name Beleodaq and was previously known as PXD101. Belinostat is disclosed in US2004/0077726 A1, its synthesis and characterisation are described in example 7.

Panobinostat (sold under the name Farydak) is an orally bioavailable HDAC inhibitor approved by the FDA (2015) and EMA (2015) for MM (Bondarev 2021). Panobinostat is investigated in patients with several hematological malignancies including AML, lymphoma, leukaemia, MCL, MM, CTCL, PTCL, NK/T-cell lymphoma, ALL, DBCL, CML, MF (Myelofibrosis), and myelodysplastic syndromes. The most common toxicities observed for Panobinostat are hypophosphatemia, hypokalemia, hyponatremia, increased creatinine, thrombocytopenia, lymphopenia, leukopenia, neutropenia, anaemia, diarrhea, fatigue, nausea, peripheral edema, decreased appetite, pyrexia, and vomiting.

Panobinostat has the following structure:

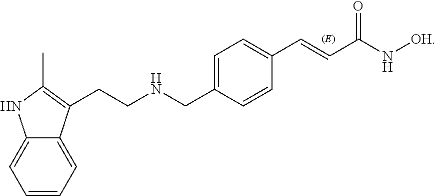 [SMILES: Cc1[nH]c2ccccc2c1CCNCc1ccc(/C=C/C(=O)NO)cc1]

The IUPAC name of Panobinostat is (2E)-N-hydroxy-3-[4-({[2-(2-methyl-1H-indol-3-yl)ethyl]amino}methyl)phenyl]acrylamide. A method to synthesise this compound and it's characterisation are described in example 200 of WO02/22577.

Tucidinostat, also referred to as chidamide, is an orally bioavailable HDAC inhibitor that was approved by China's National Medical Products Administration in 2014 for use in PTCL (Bondarev 2021). The most common toxicities observed for Tucidinostat are thrombocytopenia, leukopenia, neutropenia, QTc interval prolongation, fatigue, anorexia, diarrhea, nausea increased alanine aminotransferase levels, increased γ-glutamyltransferase levels, pulmonary infection, increased aspartate aminotransferase levels and vomiting.

Tucidinostat has the following structure;

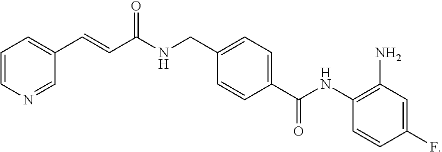 [SMILES: Nc1cc(F)ccc1NC(=O)c1ccc(CNC(=O)/C=C/c2cccnc2)cc1]

The IUPAC name of Tucidinostat is N-(2-Amino-4-fluorophenyl)-4-[[[(E)-3-pyridin-3-ylprop-2-enoyl]amino]methyl]benzamide. Tucidinostat is sold under the brand names Epidaza and Hiyasta.

Romidepsin is an intravenously administered cyclic depsipeptide approved by the FDA in 2009 for CTCL and in 2011 for PTCL (Bondarev 2021). The most common toxicities observed for Romidepsin are nausea, fatigue, infections, vomiting, anorexia, anaemia, thrombocytopenia, ECG T-wave changes, neutropenia, and lymphopenia.

Romidepsin has the following structure:

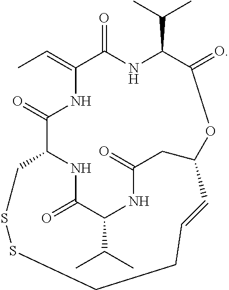 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

The IUPAC name of Romidepsin is (1S,4S,7Z,10S,16E,21R)-7-Ethylidene-4,21-diisopropyl-2-oxa-12,13-dithia-5,8,20,23-tetrazabicyclo[8.7.6]tricos-16-ene-3,6,9,19,22-pentone. Romidepsin is also known as Istodax. The synthesis of Romidepsin is disclosed in Li 1996.

Abexinostat is an orally bioavailable experimental drug candidate for cancer and has been used in trials studying the treatment of Sarcoma, Lymphoma, Leukemia, Lymphocytic, and Hodgkin Disease, among others. Abexinostat has the following structure:

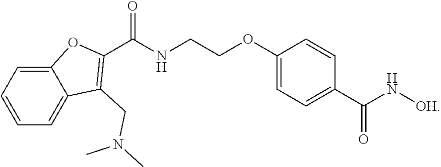 [SMILES: CN(C)Cc1c(C(=O)NCCOc2ccc(C(=O)NO)cc2)oc2ccccc12]

The IUPAC name of Abexinostat is 3-[(Dimethylamino)methyl]-N-{2-[4-(hydroxycarbamoyl) phenoxy]ethyl}-1-benzofuran-2-carboxamide.

Pracinostat is an orally bioavailable HDAC inhibitor with potential antineoplastic activity which is currently in clinical trials for acute myeloid Leukemia. Pracinostat has the following structure:

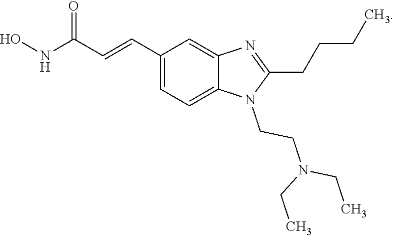 [SMILES: CCCCc1nc2cc(/C=C/C(=O)NO)ccc2n1CCN(CC)CC]

The IUPAC name of Pracinostat is (E)-3-[2-butyl-1-[2-(diethylamino)ethyl]benzimidazol-5-yl]-N-hydroxyprop-2-enamide. The dosing schedule of Pracinostat is 60 mg per day.

Quisinostat is an experimental drug candidate for the treatment of cancer. Quisinostat has been used in trials studying the treatment of Lymphoma, Neoplasms, Myelodysplastic Syndromes, and Advanced or Refractory Leukemia. Quisinostat has the following structure:

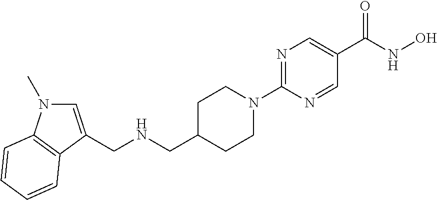 [SMILES: Cn1cc(CNCC2CCN(c3ncc(C(=O)NO)cn3)CC2)c2ccccc21]

The IUPAC name of Quisinostat is N-Hydroxy-2-[4-({[(1-methyl-1H-indol-3-yl)methyl]amino}methyl)-1-piperidinyl]-5-pyrimidinecarboxamide.

Tefinostat is a HDAC inhibitor with potential antineoplastic activity and is under investigation in a clinical trial NCT02759601 for the treatment of liver cancer. Tefinostat has the following structure:

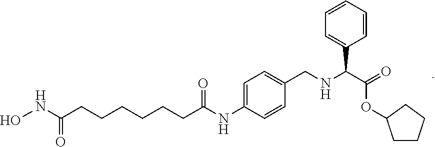 [SMILES: O=C(CCCCCCC(=O)Nc1ccc(CN[C@H](C(=O)OC2CCCC2)c2ccccc2)cc1)NO]

The IUPAC name of Tefinostat is cyclopentyl(2S)-2-[[4-[[8-(hydroxyamino)-8-oxooctanoyl]amino]phenyl]methylamino]-2-phenylacetate.

Givinostat (also known as gavinostat or ITF-2357) is a HDAC inhibitor which is in clinical trials to treat numerous cancers. Givinostat has the following structure:

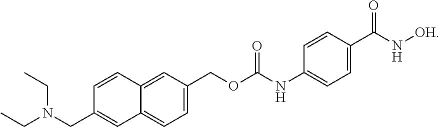 [SMILES: CCN(CC)Cc1ccc2cc(COC(=O)Nc3ccc(C(=O)NO)cc3)ccc2c1]

The IUPAC name of Givinostat is {6-[(diethylamino)methyl]naphthalen-2-yl}methyl[4-(hydroxycarbamoyl)phenyl]carbamate.

Entinostat (also known as SNDX-275 or MS-275) is a HDAC inhibitor which is undergoing clinical trials for various cancers. The structure of Entinostat is

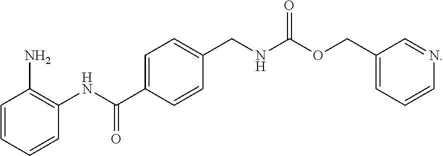 [SMILES: Nc1ccccc1NC(=O)c1ccc(CNC(=O)OCc2cccnc2)cc1]

The IUPAC name of Entinostats is (Pyridin-3-yl)methyl({4 [2-aminophenyl) carbamoyl]phenyl}methyl) carbamate.

Trapoxin ((cyclo-(L-phenylalanyl-L-phenylalanyl-D-pipecolinyl-L-2-amino-8-oxo-9,10-epoxy-decanoyl)) is a cyclotetrapeptide isolated from the fungus Helicoma ambiens. Trapoxin is a HDAC inhibitor. Trapoxin has two forms A and B.

Trapoxin A has the structure:

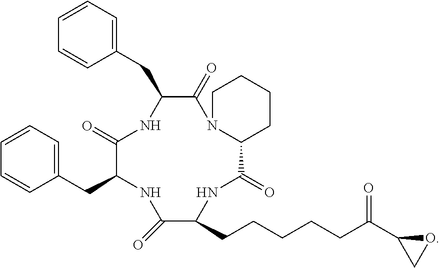 [SMILES: O=C1N[C@@H](Cc2ccccc2)C(=O)N2CCCC[C@@H]2C(=O)N[C@@H](CCCCCC(=O)[C@@H]2CO2)C(=O)N[C@H]1Cc1ccccc1]

Trapoxin B has the structure:

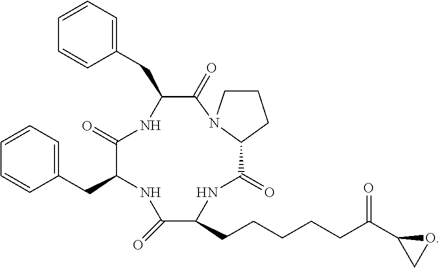 [SMILES: O=C1N[C@@H](Cc2ccccc2)C(=O)N2CCC[C@@H]2C(=O)N[C@@H](CCCCCC(=O)[C@@H]2CO2)C(=O)N[C@H]1Cc1ccccc1]

Valproic acid (also known as Valproate, sodium valproate and valproate semisodium) is a well-established therapy for seizures and bipolar disorder and has also been shown to be a HDAC inhibitor. Valproic acid first came into medical use in the 1960s and is widely genericized and is on the world health organizations list of essential medicines. Valproic acid has the structure:

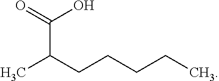

Valproic acid has the IUPAC name 2-propylpentanoic acid.

Mocetinostat (also known as MDCD0103) is a HDAC inhibitor which is undergoing clinical trials for the treatment of various cancers including follicular lymphoma, Hodgkin's lymphoma and acute myelogeneous leukemia. Clinical and pharmacodynamic data support a three-times-weekly administration at a 90 mg fixed dose. Mocetinostat displays promising antitumor activity in several hematological diseases.

Mocetinostat has the following structure:

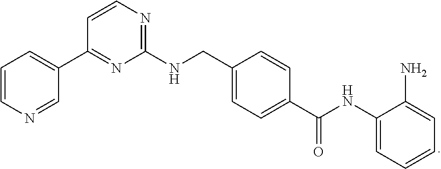 [SMILES: Nc1ccccc1NC(=O)c1ccc(CNc2nccc(-c3cccnc3)n2)cc1]

The IUPAC name of Mocetinostat is N-(2-Aminophenyl)-4-({[4-(pyridin-3-yl)pyrimidin-2-yl]amino}methyl)benzamide.

In some embodiments, the further chemotherapeutic agent may be a second HDAC inhibitor.

In other embodiments, the further chemotherapeutic agent may be a DNA methyltransferase inhibitor. DNA methylation mediated by DNA methyltransferase is an important epigenetic process regulating gene expression and plays a key role in silencing tumor suppressor genes in cancer. Hence it has become a promising therapeutic target for cancer treatment especially for haematological tumours (Zhang 2022), DNMT inhibitors can also enhance the immunogenicity of tumour cells by promoting tumour antigen presentation or enhancing the function of cytotoxic T cells. Thus DNMT is also a rational therapeutic target in haematological malignancies. First generation DNMT inhibitors are however characterized by high toxicity, poor selectivity and low bioavailability.

DNMT inhibitors include but are not limited to 5-azacitidine (azacytidine (AZA)), 5-aza-2′-deoxycytidine (decitabine (DAC)), clofarabine, gaudecitabine and GSK3685032. 5-azacitidine (azacytidine (AZA)) has the structure:

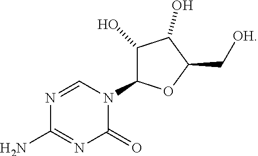 [SMILES: Nc1ncn([C@@H]2O[C@H](CO)[C@@H](O)[C@H]2O)c(=O)n1]

5-Azacitidine is a cytarabine derivative synthesized in 1964, and was first approved by the FDA in 2004.

Decitabine has the structure:

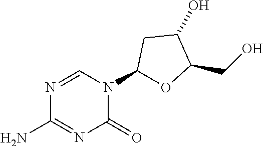 [SMILES: Nc1ncn([C@H]2C[C@H](O)[C@@H](CO)O2)c(=O)n1]

Decitabine was approved by the FDA in 2006. The inhibitory activity of DNMT is 30 times more than that of azacytidine (Zhang 2022).

Clofarabine has the structure:

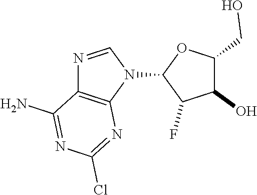 [SMILES: Nc1nc(Cl)nc2c1ncn2[C@@H]1O[C@H](CO)[C@@H](O)[C@@H]1F]

Clofarabine is a purine nucleoside DNMTi, which was approved by the FDA in 2004.

Guadecitabine (SGI-110) has the structure:

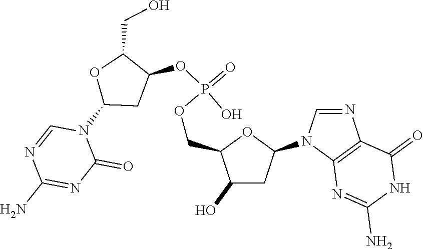 [SMILES: Nc1ncn([C@H]2C[C@H](OP(=O)(O)OC[C@H]3O[C@@H](n4cnc5c(=O)[nH]c(N)nc54)C[C@H]3O)[C@@H](CO)O2)c(=O)n1]

is a dinucleotide derivative of decitabine.

GSK3685032 has the structure:

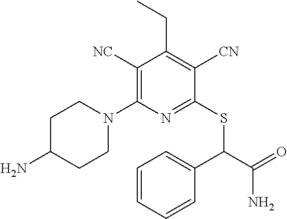 [SMILES: CCc1c(C#N)c(SC(C(N)=O)c2ccccc2)nc(N2CCC(N)CC2)c1C#N]

and is described in Pappalardi 2021. It acts as a competitive inhibitor of DNMT1 via competition with the DNMT1 active-site loop and target-recognition domain to incorporate into the hemi-methylated DNA.

Other DNMT inhibitors include, but are not limited to: RX-3117 (TV-1360), 5-fluoro-2-deoxycytidine, 6-dihydro-5-azacytidine (DHAC), fazarabine, cladribine, fludarabine, procaine, epgallocatechin gallate (EGCG), hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UVI5008.

### Definitions

The language “pharmaceutical composition” includes compositions comprising an active ingredient and a pharmaceutically acceptable excipient, carrier or diluent, wherein the active ingredient is Compound 1 or a pharmaceutically acceptable salt thereof, or a HDAC inhibitor or a pharmaceutically acceptable salt thereof, or the further chemotherapeutic agent. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders or sachets. In some embodiments, the pharmaceutical compositions are in the form of a sterile injectable solution in one or more aqueous or non-aqueous non-toxic parenterally acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The language “treat,” “treating” and “treatment” includes the reduction or inhibition of tumour cells of a haematological malignancy in a subject, amelioration of one or more symptoms of a haematological malignancy in a subject, or the slowing or delaying of progression of a haematological malignancy in a subject. The language “treat,” “treating” and “treatment” also includes the reduction or inhibition of the growth of a tumor or proliferation of cancerous cells in a subject.

The language “inhibit”, “inhibition” or “inhibiting” includes a decrease in the baseline activity of a biological activity or process.

The term “subject” includes warm-blooded mammals, for example, primates, dogs, cats, rabbits, rats, and mice. In some embodiments, the subject is a primate, for example, a human. In some embodiments, the subject is suffering from a haematological malignancy.

The language “therapeutically effective amount” includes that amount of Compound 1 and that amount of a HDAC inhibitor which together will elicit a biological or medical response in a subject, for example, the reduction or inhibition of tumour cells; amelioration of symptoms of a haematological malignancy; or the slowing or delaying of progression of a haematological malignancy. In some embodiments, the language “therapeutically effective amount” includes the amount of Compound 1 and a HDAC inhibitor together that is effective to at least partially alleviate, inhibit, and/or ameliorate a haematological malignancy or inhibit tumour cells and/or reduce or inhibit the proliferation of cancerous cells in a subject. In some embodiments, the language “therapeutically effective amount” includes the amount of Compound 1, the HDAC inhibitor and the further chemotherapeutic agent together that is effective to at least partially alleviate, inhibit, and/or ameliorate a haematological malignancy or inhibit tumour cells and/or reduce or inhibit the proliferation of cancerous cells in a subject.

In some embodiments, disclosed is a method of treating a haematological malignancy in a subject in need thereof, comprising administering to the subject a first amount of Compound 1 or a pharmaceutically acceptable salt thereof, and a second amount of a HDAC inhibitor or a pharmaceutically acceptable salt thereof. In the method, the first amount and the second amount together comprise a therapeutically effective amount. In some of these embodiments, a third amount of a further chemotherapeutic agent is administered.

In some embodiments, disclosed is Compound 1, or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a HDAC inhibitor or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is a HDAC inhibitor, or a pharmaceutically acceptable salt thereof, for use in the treatment of a haematological malignancy in a subject, wherein said treatment comprises the separate, sequential or simultaneous administration of i) said HDAC inhibitor, or a pharmaceutically acceptable salt thereof, and ii) Compound 1, or a pharmaceutically acceptable salt thereof, to said subject. In some of these embodiments, the treatment further comprises the separate, sequential or simultaneous administration of iii) a further chemotherapeutic agent, to said subject.

In some embodiments, disclosed is the use of Compound 1, or a pharmaceutically acceptable salt thereof, in the manufacture of a medicament for use in the treatment of a haematological malignancy in a subject, wherein said treatment comprises the separate, sequential or simultaneous administration of i) said medicament comprising Compound 1, or a pharmaceutically acceptable salt thereof, and ii) a HDAC inhibitor, or a pharmaceutically acceptable salt thereof, to said subject. In some of these embodiments, the treatment further comprises the separate, sequential or simultaneous administration of iii) a further chemotherapeutic agent, to said subject.

In some embodiments, Compound 1 or a pharmaceutically acceptable salt thereof and a HDAC inhibitor or a pharmaceutically acceptable salt thereof (and the further chemotherapeutic agent, if present) are administered separately, sequentially or simultaneously in a treatment cycle. In some embodiments, Compound 1 or a pharmaceutically acceptable salt thereof is continuously administered in the treatment cycle and a HDAC inhibitor or a pharmaceutically acceptable salt is also continuously administered in the treatment cycle. In some of these embodiments, the further chemotherapeutic agent is also continuously administered in the treatment cycle.

In some embodiments the HDAC inhibitor is selected from Abexinostat (PCI-24781), Pracinostat (SB939), Quisinostat (JNJ-26481585), Tefinostat (CHR-2845), Panobinostat (LBH589), Belinostat (PXD101), Givinostat (ITF2357), Tucidinostat (CS-055, HBI-8000), Vorinostat (suberoylanilide hydroxamic acid, SAHA), Mocetinostat (MGCD0103), Valproic acid (VAL), Entinostat (MS275), Romidepsin (depsipeptide, FK228), and Trapoxin (TPX).

In some embodiments the HDAC inhibitor is selected from Vorinostat, Belinostat Panobinostat, Tucidinostat or Romidepsin.

In further embodiments the HDAC inhibitor is selected from Vorinostat or Romidepsin.

The term “continuous” or “continuously” refers to administration of a therapeutic agent, e.g. Compound 1, at regular intervals without stopping or interruption, i.e., no void day. By “void day”, it is meant a day when a therapeutic agent is not administered.

A “cycle”, “treatment cycle” or “dosing schedule”, as used herein, refers to a period of combination treatment that is repeated on a regular schedule. For example, the treatment can be given for one week, two weeks, or three weeks wherein Compound 1 and a HDAC inhibitor are administered in a coordinated fashion. In some embodiments, a treatment cycle is about 1 week to about 3 months. In some embodiments, a treatment cycle is about 5 days to about 1 month. In some embodiments, a treatment cycle is about 1 week to about 3 weeks. In some embodiments, a treatment cycle is about 1 week, about 10 days, about 2 weeks, about 3 weeks, about 4 weeks, about 2 months, or about 3 months.

In some embodiments, Compound 1 or a pharmaceutically acceptable salt thereof and a HDAC inhibitor or a pharmaceutically acceptable salt thereof (and the further chemotherapeutic agent, if present) are administered to the human subject in one or more treatment cycles, e.g., a treatment course. A “treatment course” comprises multiple treatment cycles, which can be repeated on a regular schedule, or adjusted as a tapered schedule as the patient's disease progression is monitored. For example, a patient's treatment cycles can have longer periods of treatment and/or shorter periods of rest at the beginning of a treatment course (e.g., when the patient is first diagnosed), and as the cancer enters remission, the rest period lengthens, thereby increasing the length of one treatment cycle. The period of time for treatment and rest in a treatment cycle, the number of treatment cycles, and the length of time for the treatment course can be determined and adjusted throughout the treatment course by the skilled artisan based on the patient's disease progression, treatment tolerance, and prognosis. In some embodiments, the method comprises 1 to 10 treatment cycles. In some embodiments, the method comprises 2 to 8 treatment cycles.

In some embodiments, Compound 1 or a pharmaceutically acceptable salt thereof is administered for 28 days in a 28-day treatment cycle, and a HDAC inhibitor or a pharmaceutically acceptable salt thereof is administered for 28 days in a 28-day treatment cycle.

In some embodiments, Compound 1 or a pharmaceutically acceptable salt thereof is administered for 28 days in a 28-day treatment cycle, and a HDAC inhibitor or a pharmaceutically acceptable salt thereof is administered on days 1, 8 and 15 of a 28-day treatment cycle.

In some embodiments, Compound 1 or a pharmaceutically acceptable salt thereof is administered for 28 days in a 28-day treatment cycle, and a HDAC inhibitor or a pharmaceutically acceptable salt thereof is administered on days 1, 3, 8, 10 and 12 of a 21-day treatment cycle.

In some embodiments, Compound 1 or a pharmaceutically acceptable salt thereof is administered for 28 days in a 28-day treatment cycle, and a HDAC inhibitor or a pharmaceutically acceptable salt thereof is administered on days 1 to 5 of a 21-day treatment cycle.

### Dosage

In some embodiments, Compound 1 or a pharmaceutically acceptable salt thereof is administered orally. In some embodiments, Compound 1 or a pharmaceutically acceptable salt thereof is in tablet dosage form. In some embodiments, Compound 1 as the hemi-fumarate salt is administered in a dose of 40 mg per day. In some embodiments Compound 1 as the hemi-fumarate salt is administered in a dose of 80 mg per day.

In some embodiments, a HDAC inhibitor or a pharmaceutically acceptable salt thereof is administered orally. In some embodiments, a HDAC inhibitor or a pharmaceutically acceptable salt thereof is in tablet dosage form. In some embodiments, a HDAC inhibitor or a pharmaceutically acceptable salt thereof is in capsule dosage form. In some embodiments, a HDAC inhibitor or a pharmaceutically acceptable salt thereof is administered in a dose between 10 mg and 1 g per day.

In some embodiments, a HDAC inhibitor or a pharmaceutically acceptable salt thereof is administered as an intravenous (IV) infusion.

In some embodiments, the further chemotherapeutic agent is administered orally. In some embodiments, the further chemotherapeutic agent is in tablet dosage form. In some embodiments, the further chemotherapeutic agent is in capsule dosage form. In some embodiments, the further chemotherapeutic agent is administered by injection. In some embodiments the further chemotherapeutic agent is administered by intravenous (IV) infusion. In some embodiments, the further chemotherapeutic agent is administered in a dose between 10 mg and 1 g per day.

In some embodiments, Compound 1 and a HDAC inhibitor are taken together on an empty stomach, with no food two hours before, and one hour after.

In some embodiments, Compound 1 is taken on an empty stomach, with no food two hours before, and one hour after, and the HDAC inhibitor is taken with food, so either at least two hours before, or one hour after Compound 1.

In some embodiments, the HDAC inhibitor is dosed weekly.

In some embodiments, disclosed is a pharmaceutical product comprising i) Compound 1 or a pharmaceutically acceptable salt thereof, and ii) a HDAC inhibitor or a pharmaceutically acceptable salt thereof. In some embodiments, Compound 1 or a pharmaceutically acceptable salt thereof, and a HDAC inhibitor or a pharmaceutically acceptable salt thereof are present in a single dosage form. In some embodiments, Compound 1 or a pharmaceutically acceptable salt thereof, and a HDAC inhibitor or a pharmaceutically acceptable salt thereof are present separate dosage forms. In some of these embodiments, the pharmaceutical product further comprises iii) a further chemotherapeutic agent.

In some embodiments, disclosed is a kit comprising: a first pharmaceutical composition comprising Compound 1, or a pharmaceutically acceptable salt thereof; a second pharmaceutical composition comprising a HDAC inhibitor, or a pharmaceutically acceptable salt thereof; and instructions for using the first and second pharmaceutical compositions in combination. In some of these embodiments, the kit further comprises a third pharmaceutical composition comprising a further chemotherapeutic agent, and the instructions are for using the first, second and third pharmaceutical compositions in combination.

In some embodiments, the HDAC inhibitor is Abexinostat (PCI-24781), Pracinostat (SB939), Quisinostat (JNJ-26481585), Tefinostat (CHR-2845), Panobinostat (LBH589), Belinostat (PXD101), Givinostat (ITF2357), Tucidinostat (CS-055, HBI-8000), Vorinostat (suberoylanilide hydroxamic acid, SAHA), Mocetinostat (MGCD0103), Valproic acid (VAL), Entinostat (MS275), Romidepsin (depsipeptide, FK228), and Trapoxin (TPX).

In some embodiments the HDAC inhibitor is Vorinostat. Vorinostat as a monotherapy is administered orally for example by tablets or capsules. The dosing schedule for Vorinostat is 400 mg or 300 mg once daily taken with food and Vorinistat capsules come in the strength of 100 mg. In some embodiments the Vorinostat is administered at a dose of 100 mg-500 mg per day. In some embodiments the Vorinostat is administered at a dose of 200 mg, 300 mg or 400 mg per day. In some embodiments the Vorinostat is administered at a dose of 300 mg per day. In some embodiments the Vorinostat is administered at a dose of 400 mg per day. In some embodiments the dosage of Vorinostat is administered orally by 3 or 4 100 mg capsules per day. In further embodiments Vorinostat is administered daily with food.

In some embodiments, disclosed is a pharmaceutical product comprising i) Compound 1, or a pharmaceutically acceptable salt thereof, and ii) Vorinostat, or a pharmaceutically acceptable salt thereof. In some embodiments Compound 1 or a pharmaceutically acceptable salt thereof and Vorinostat, or a pharmaceutically acceptable salt thereof are present in a single dosage form. In some embodiments Compound 1 or a pharmaceutically acceptable salt thereof and Vorinostat, or a pharmaceutically acceptable salt thereof are present as separate dosage forms.

In some embodiments the HDAC inhibitor is Belinostat. Belinostat is administered intravenously. The recommended dosage of Belinostat is 1000 mg per square meter body surface administered over 30 minutes by intravenous infusion once daily on days 1-5 of a 21-day cycle. Cycles can be repeated until disease progression or unacceptable toxicity. Dose adjustments for thrombocytopenia and neutropenia on platelet and absolute neutrophil nadir counts are common. Belinostat for injection comes in the form of a vial with 500 mg lyophilized powder for reconstitution. In some embodiments the Belinostat is administered at a dosage of 500-1200 mg per square meter of body surface by intravenous infusion over 20-40 minutes once daily. In further embodiments Belinostat is administered at a dosage of 500-1200 mg per square meter of body surface by intravenous infusion over 20-40 minutes once daily on days 1 to 5 of a 21-day cycle.

In some embodiments the HDAC inhibitor is Panobinostat. Panobinostat is administered orally for example by tablets or capsules. The recommended starting dose of panobinostat is 20 mg, taken orally once a day, on days 1, 3, 5, 8, 10 and 12 of a 21-day cycle. Panibostat capsules come in three strengths (10, 15, and 20 mg). In some embodiments Panobinostat is administered at a dose of 5-40 mg once per day. In some embodiments Panobinostat is administered at a dose of 20 mg once per day. In some embodiments Panobinostat is administered at a dose of 5-40 mg once per day every 2-3 days for 2 weeks. In further embodiments Panobinostat is administered at a dose of 5-40 mg once per day on days 1, 3, 5, 8, 10 and 12 of a 21-day cycle.

In some embodiments the HDAC inhibitor is Tucidinostat. Tucidinostat is administered orally for example by tablets or capsules. The recommended dose and dose schedule for Tucidinostat is 40 mg twice weekly. In some embodiments Tucidinostat is administered at a dose of 20-60 mg twice weekly. In some embodiments Tucidinostat is administered at a dose of 40 mg twice weekly. In some embodiments 40 mg of Tucidinostat is administered once every 3 days.

In some embodiments the HDAC inhibitor is Romidepsin. Romidepsin is administered intravenously. The recommended dose and dose schedule for Romidepsin is 14 mg per square meter body surface administered over a four-hour period on days 1, 8 and 15 of a 28-day cycle. Treatment discontinuation or interruption with or without dose reduction to 10 mg per square meter body surface may be needed to manage adverse drug reactions. Romidepsin for injection comes in the form of a vial with 10 mg for reconstitution. In some embodiments Romidepsin is administered at a dose of 5-30 mg per square meter body surface administered over a 2-to-6-hour period. In further embodiments Romidepsin is administered at a dose of 5-30 mg per square meter body surface administered once a week. In further embodiments Romidepsin is administered at a dose of 5-30 mg per square meter body surface administered over a 2-to-6-hour period on days 1, 8 and 15 of a 28-day cycle. In some embodiments Romidepsin is administered at a dose of 10 mg or 14 mg per square meter body surface administered over a 2-to-6-hour period. In further embodiments the Romidepsin is administered once weekly. In further embodiments Romidepsin is administered over about a 4 hour period.

In some embodiments, disclosed is a pharmaceutical product comprising i) Compound 1, or a pharmaceutically acceptable salt thereof, and ii) Romidepsin or a pharmaceutically acceptable salt thereof. In some embodiments Compound 1 or a pharmaceutically acceptable salt thereof and Romidepsin or a pharmaceutically acceptable salt thereof are present as separate dosage forms. In some embodiments Compound 1 is administered orally as a tablet or a capsule and Romidepsin is administered intravenously. In some embodiments the oral and intravenous administration is sequential or simultaneous administration.

In some embodiments the haematological malignancy is lymphoma, including cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.

In some embodiments the haematological malignancy is leukemia, including ALL, AML, CLL, chronic myelogenous leukemia, hairy cell leukemia, myelodysplastic syndromes, myeloproliferative disorders and myelofibrosis.

In some embodiments the haematological malignancy is myeloma, including multiple myeloma.

Without wishing to be bound by theory, the combination of Compound 1 and a HDAC inhibitor may be beneficial for patients not responding or refractory to either Compound 1 or the HDAC inhibitor. In addition the combination of Compound 1 and a HDAC inhibitor may deepen the response to either Compound 1 or the HDAC inhibitor or make the response more durable. Furthermore, the combination of Compound 1 and a HDAC inhibitor may allow for dose reductions of the HDAC inhibitor resulting in improved tolerability and quality of life.

Furthermore, where the further chemotherapeutic agent is a second HDAC inhibitor, the reasoning above may equally apply. Where the further chemotherapeutic agent is a a DNA methyltransferase inhibitor, the combination may be beneficial for patients not responding or refractory to any of Compound 1, the HDAC inhibitor and the DNMT inhibitor. In addition, the combination of Compound 1, the HDAC inhibitor and the DNMT inhibitor may deepen the response to Compound 1, the HDAC inhibitor or the DNMT inhibitor or make the response more durable. Furthermore, the combination of Compound 1, the HDAC inhibitor and the DNMT inhibitor may allow for dose reductions of the HDAC inhibitor or DNMT inhibitor resulting in improved tolerability and quality of life.

### Methods of the Invention

As described in more detail below, the present inventors have surprisingly found that the combination of Compound 1 with HDAC inhibitors has synergistic effects on the proliferation of heamatological malignancy cell lines. Thus, a combination therapy with improved antiproliferative activity can be provided.

Secondly, the present inventors surprisingly found that the synergistic anti-tumour activity of the combination of Compound 1 with HDAC inhibitors is not limited to a specific heamatological malignancy. Thus, a combination therapy with improved therapeutic activity across multiple heamatological malignancies can be provided.

Additionally, the present inventors surprisingly found that the combination of Compound 1 with HDAC inhibitors results in tumour cell killing that is not observed with either inhibitor alone. Thus, a combination that can reduce tumour burden can be provided.

Furthermore, the present inventors surprisingly found that the combination of Compound 1 with HDAC inhibitors has synergistic effects already at sub-therapeutic doses. Thus, a combination with improved tolerability may be provided.

### EXAMPLES

The compounds of the application will now be further explained by reference to the following non-limiting examples.

### Example 1. Efficacy of Compound 1 Combined with HDAC Inhibitors Vorinostat and Romidepsin in an In Vitro Assay Using the Cutaneous T-Cell Lymphoma (CTCL) Cell Line HH

HH (CRL-2105) cells were exposed to increasing doses of Compound 1, to increasing doses of Vorinostat or Romidepsin, and to increasing doses of the combination of Compound 1 and Vorinostat or Romidepsin. Compound 1 and Vorinostat were used at a maximum concentration of 10 UM, following an eight fold dose response design based on 1:3 compound dilutions plus an untreated control. Romidepsin was used at a maximum concentration of 20 UM, following an eight fold dose response design based on 1:2 compound dilutions plus an untreated control.

Cells were incubated for 72 hours at 37° C. and 5% CO₂. The antiproliferative effect of single and combination treatments were determined by adding 20 μL MTT [3-(4,5-dimethylthiazolyl-2)-2,5-diphenyltetrazoliumbromide] reagent (Sigma Aldrich, Buchs, Switzerland) to each well and the plates were incubated at 37° C. for 4 hours, followed by adding 50 μL of sodium dodecyl sulfate (SDS) (250 μM SDS, 0.21% fuming HCl) lysis buffer. The lysed cells were kept overnight, and then the absorbance was read at 570 nm using the Cytation 3 instrument (BioTek, Winoosku, VT, USA). The effect of the combinations was determined according to the Chou-Talalay Combination Index (CI) (Chou 2008, Chou 2010) and calculated with the Synergy R package (Lee 2007). The effect of the combinations was defined as strongly synergistic for CI values below <0.3, synergistic for CI values between 0.3 and 0.9, additive for CI values between 0.9 and 1.1, and antagonistic for CI values >1.1.

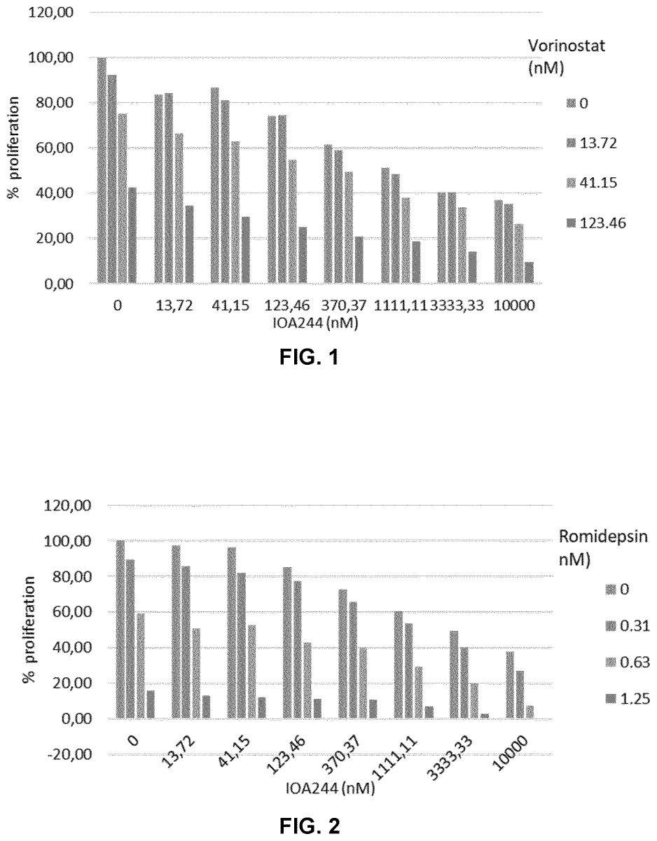

Data from two representative experiments is shown FIGS. 1 and 2.

The combination of Compound 1 and Vorinostat in HH is synergistic with a median CI value of 0.64. The combination of Compound 1 and Romidepsin in HH is synergistic with a median CI value of 0.89.

### Example 2. Efficacy of Compound 1 Combined with HDAC Inhibitors in an In Vitro Assay Using the Several Different Haematological Cell Lines

Cell lines from different haematological malignancies were exposed to increasing doses of Compound 1, to increasing doses of HDAC inhibitor, and to increasing doses of the combination of Compound 1 and HDAC inhibitor. Compound 1 and Vorinostat were used at a maximum concentration of 10 μM, following an eight fold dose response design based on 1:3 compound dilutions plus an untreated control. Belinostat was used at a maximum concentration of 10 μM, following an eight fold dose response design based on 1:4 compound dilutions plus an untreated control.

Cells were incubated for 72 hours at 37° C. and 5% CO₂. The antiproliferative effect of single and combination treatments were determined by adding 20 μL MTT [3-(4,5-dimethylthiazolyl-2)-2,5-diphenyltetrazoliumbromide] reagent (Sigma Aldrich, Buchs, Switzerland) to each well and the plates were incubated at 37° C. for 4 hours, followed by adding 50 μL of sodium dodecyl sulfate (SDS) (250 UM SDS, 0.21% fuming HCl) lysis buffer. The lysed cells were kept overnight, and then the absorbance was read at 570 nm using the Cytation 3 instrument (BioTek, Winoosku, VT, USA). The effect of the combinations was determined according to the Chou-Talalay Combination Index (CI) (Chou 2008, Chou 2010) and calculated with the Synergy R package (Lee 2007). The effect of the combinations was defined as strongly synergistic for CI values below <0.3, synergistic for CI values between 0.3 and 0.9, additive for CI values between 0.9 and 1.1, and antagonistic for CI values >1.1.

CI values from representative experiments is shown in Table 1:

TABLE 1
CI values for the combination of Compound 1 and HDAC inhibitors
in different haematological cell lines

| | | Cl value | 95% Cl confidence |
|---|---|---|---|
| HDAC inhibitor | Cell line (origin) | (median) | interval |
| Belinostat | Farage (DLBCL) | 0.51 | 0.46-0.65 |
| Belinostat | JVM2 (MCL) | 0.62 | 0.44-1.1 |
| Belinostat | TMD8 (DLBCL) | 0.87 | 0.62-1.2 |
| Belinostat | A20 (murine B- | 0.87 | 0.45-1.3 |
| | cell lymphoma) | | |
| Belinostat | MEC1 (CLL) | 0.45 | 0.36-0.67 |
| Vorinostat | Farage (DLBCL) | 0.69 | 0.58-0.92 |
| Vorinostat | JVM2 (MCL) | 0.71 | 0.58-0.82 |
| Vorinostat | MEC1 (CLL) | 0.83 | 0.66-1.49 |

### REFERENCES

A number of publications are cited above in order to more fully describe and disclose the invention and the state of the art to which the invention pertains. Full citations for these references are provided below. The entirety of each of these references is incorporated herein.

| | Reference | Doi |
|---|---|---|
| Bondarev 2021 | Bondarev, AD, et al., Br J Clin | 10.1111/bcp. 14889 |
| | Pharmacol., 2021 Dec; 87(12): | |
| | 4577-4597 | |
| Carlo-Stella | Carlo-Stella, C. et al., EHA | |
| 2022 | 2022, poster 1116 | |
| Chou 2008 | Chou, T., Leukemia & | 10.1080/ |
| | Lymphoma (2008), 49(11) | 10428190802353591 |
| Chou 2010 | Chou, T., Cancer Res (2010), | 10.1158/0008-5472.CAN- |
| | 70 (2), 440-446 | 09-1947 |
| Haselmayer | Haselmayer, P. et al., Frontiers | 10.3389/fimmu.2014.00233 |
| 2014 | in Immunology (2014), Vol. 5, | |
| | Art. 233, p. 1-15 | |
| Intlekofer and | Intlekofer, M. and Younes, A. | 10.1038/nrclinonc.2014.137 |
| Younes 2014 | Nature reviews Clinical | |
| | Oncology (2014), Vol. 11, p. | |
| | 585-596 | |
| Jephcote 2020 | Jephcote, C. et al., | 10.1186/s12940-020- |
| | Environmental Health (2020), | 00582-1 |
| | Vol. 19:53 | |
| Lee 2007 | Lee, J. et al., J Biopharm Stat | 10.1080/ |
| | (2007), 17(3),461-80 | 10543400701199593 |
| Li 1996 | Li, KW., et al., Journal of the | 10.1021/ja9613724 |
| | American Chemical Society | |
| | 1996 118 (30), 7237-7238 | |
| Richardson | Richardson, N. The Lancet | 10.1016/S1470- |
| 2022 | Oncology (2022), Vol. 23, p. | 2045(22)00200-5 |
| | 563-566 | |
| Tarantelli 2022 | Tarantelli, C. et al. ENA 2022, | |
| | poster 223 | |

### Statements

1. A method of treating a haematological malignancy in a subject in need thereof, comprising administering to the subject a first amount of a compound of Formula I:

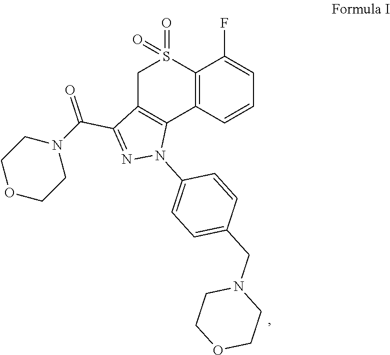 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and a second amount of a HDAC inhibitor or a pharmaceutically acceptable salt thereof, wherein the first amount and the second amount together comprise a therapeutically effective amount.
2. The method according to statement 1, wherein the haematological malignancy is lymphoma, leukaemia, myeloma, myelodysplastic syndrome or myeloproliferative diseases.
3. The method of statement 2, wherein the lymphoma is cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.
4. The method of statement 2, wherein the leukemia is ALL, AML, CLL, chronic myelogenous leukemia, hairy cell leukemia, myelodysplastic syndromes, myeloproliferative disorders and myelofibrosis.
5. The method of any one of statements 1 to 4, wherein the HDAC inhibitor is selected from Abexinostat, Pracinostat, Guisinostat, Tefinostat, Panobinostat, Belinostat, Givinostat, Tucidinostat, Vorinostat, Mocetinostat, Valproic acid, Entinostat, Romidepsin, and Trapoxin.
6. The method according to any of statements 1 to 5, wherein Compound 1 is administered as the hemifumarate salt in a dose of 40 mg or 80 mg per day.
7. The method according to any one of statements 1 to 6, wherein a HDAC inhibitor or a pharmaceutically acceptable salt thereof is administered in a dose between 10 mg and 1 g per day.
8. The method according to any one of statements 1 to 7, wherein the HDAC inhibitor is selected from Vorinostat or a pharmaceutically acceptable salt thereof.
9. The method according to any one of statements 1 to 8, wherein the HDAC inhibitor is Vorinostat or a pharmaceutically acceptable salt thereof and is administered at a dose of 200 mg, 300 mg or 400 mg per day.
10. The method according to any one of statements 1 to 7, wherein the HDAC inhibitor is Romidepsin.
11. A method according to any one of statements 1 to 10, wherein a third amount of a further chemotherapeutic agent is administered.
12. A method according to statement 11, wherein the further chemotherapeutic agent is a second HDAC inhibitor.
13. A method according to statement 11, wherein the further chemotherapeutic agent is a DNA methyltransferase inhibitor.
14. A method according to statement 13, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UVI5008.
15. A compound of Formula I:

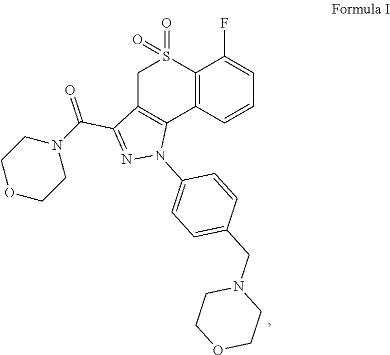 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, for use in the treatment of a haematological malignancy in a subject, wherein said treatment comprises the separate, sequential or simultaneous administration of i) said Compound 1, or a pharmaceutically acceptable salt thereof, and ii) a HDAC inhibitor, or a pharmaceutically acceptable salt thereof, to said subject.
16. Compound 1, or a pharmaceutically acceptable salt thereof, for use according to statement 15, wherein the haematological malignancy is lymphoma, leukaemia, myeloma, myelodysplastic syndrome or myeloproliferative diseases.
17. Compound 1, or a pharmaceutically acceptable salt thereof, for use according to statement 16, wherein the lymphoma is cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.
18. Compound 1, or a pharmaceutically acceptable salt thereof, for use according to statement 16, wherein the leukemia is ALL, AML, CLL, chronic myelogenous leukemia, hairy cell leukemia, myelodysplastic syndromes, myeloproliferative disorders and myelofibrosis.
19. Compound 1, or a pharmaceutically acceptable salt thereof, for use according to any one of statements 15 to 18, wherein the HDAC inhibitor is selected from Abexinostat, Pracinostat, Guisinostat, Tefinostat, Panobinostat, Belinostat, Givinostat, Tucidinostat, Vorinostat, Mocetinostat, Valproic acid, Entinostat, Romidepsin, and Trapoxin.
20. Compound 1, or a pharmaceutically acceptable salt thereof, for use according to any one of statements 15 to 19, wherein Compound 1 is administered as the hemifumarate salt in a dose of 40 mg or 80 mg per day.
21. Compound 1, or a pharmaceutically acceptable salt thereof, for use according to any one of statements 15 to 20, wherein a HDAC inhibitor or a pharmaceutically acceptable salt thereof is administered in a dose between 10 mg and 1 g per day.
22. Compound 1, or a pharmaceutically acceptable salt thereof, for use according to any one of statements 15 to 21, wherein the HDAC inhibitor is selected from Vorinostat or a pharmaceutically acceptable salt thereof.
23. Compound 1, or a pharmaceutically acceptable salt thereof, for use according to any one of statements 15 to 22, wherein the HDAC inhibitor is Vorinostat or a pharmaceutically acceptable salt thereof and is administered at a dose of 200 mg, 300 mg or 400 mg per day.
24. Compound 1, or a pharmaceutically acceptable salt thereof, for use according to any one of statements 15 to 21, wherein the HDAC inhibitor is Romidepsin.
25. Compound 1, or a pharmaceutically acceptable salt thereof, for use according to any one of statements 15 to 24, wherein the treatment further comprises the separate, sequential or simultaneous administration of iii) a further chemotherapeutic agent, to said subject.
26. Compound 1, or a pharmaceutically acceptable salt thereof, for use according to statement 25, wherein the further chemotherapeutic agent is a second HDAC inhibitor.
27. Compound 1, or a pharmaceutically acceptable salt thereof, for use according to statement 25, wherein the further chemotherapeutic agent is a DNA methyltransferase inhibitor.
28. Compound 1, or a pharmaceutically acceptable salt thereof, for use according to statement 27, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UVI5008.
29. A HDAC inhibitor, or a pharmaceutically acceptable salt thereof, for use in the treatment of a haematological malignancy in a subject, wherein said treatment comprises the separate, sequential or simultaneous administration of i) said HDAC inhibitor, or a pharmaceutically acceptable salt thereof, and ii) a compound of Formula I:

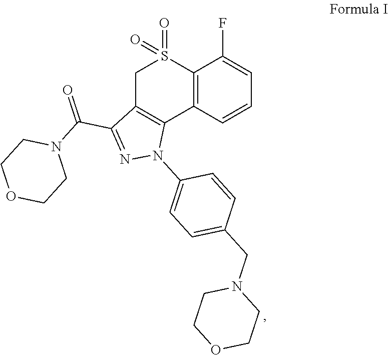 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, to said subject.
30. A HDAC inhibitor, or a pharmaceutically acceptable salt thereof, for use according to statement 29, wherein the haematological malignancy is lymphoma, leukaemia, myeloma, myelodysplastic syndrome or myeloproliferative diseases.
31. A HDAC inhibitor, or a pharmaceutically acceptable salt thereof, for use according to statement 30, wherein the lymphoma is cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.
32. A HDAC inhibitor, or a pharmaceutically acceptable salt thereof, for use according to statement 30, wherein the leukemia is ALL, AML, CLL, chronic myelogenous leukemia, hairy cell leukemia, myelodysplastic syndromes, myeloproliferative disorders and myelofibrosis.
33. A HDAC inhibitor, or a pharmaceutically acceptable salt thereof, for use according to any one of statements 29 to 32, wherein the HDAC inhibitor is selected from Abexinostat, Pracinostat, Guisinostat, Tefinostat, Panobinostat, Belinostat, Givinostat, Tucidinostat, Vorinostat, Mocetinostat, Valproic acid, Entinostat, Romidepsin, and Trapoxin.
34. A HDAC inhibitor, or a pharmaceutically acceptable salt thereof, for use according to any one of statements 29 to 33, wherein Compound 1 is administered as the hemifumarate salt in a dose of 40 mg or 80 mg per day.
35. A HDAC inhibitor, or a pharmaceutically acceptable salt thereof, for use according to any one of statements 29 to 34, wherein a HDAC inhibitor or a pharmaceutically acceptable salt thereof is administered in a dose between 10 mg and 1 g per day.
36. A HDAC inhibitor, or a pharmaceutically acceptable salt thereof, for use according to any one of statements 29 to 35, wherein the HDAC inhibitor is selected from Vorinostat or a pharmaceutically acceptable salt thereof.
37. A HDAC inhibitor, or a pharmaceutically acceptable salt thereof, for use according to any one of c statements 29 to 36, wherein the HDAC inhibitor is Vorinostat or a pharmaceutically acceptable salt thereof and is administered at a dose of 200 mg, 300 mg or 400 mg per day.
38. A HDAC inhibitor, or a pharmaceutically acceptable salt thereof, for use according to any one of statements 29 to 37, wherein the HDAC inhibitor is Romidepsin.
39. A HDAC inhibitor, or a pharmaceutically acceptable salt thereof, for use according to any one of statements 29 to 38, wherein the treatment further comprises the separate, sequential or simultaneous administration of iii) a further chemotherapeutic agent, to said subject.
40. A HDAC inhibitor, or a pharmaceutically acceptable salt thereof, for use according to statement 39, wherein the further chemotherapeutic agent is a second HDAC inhibitor.
41. A HDAC inhibitor, or a pharmaceutically acceptable salt thereof, for use according to statement 39, wherein the further chemotherapeutic agent is a DNA methyltransferase inhibitor.
42. A HDAC inhibitor, or a pharmaceutically acceptable salt thereof, for use according to statement 41, wherein the DNA methyltransferase inhibitor is selected from 5-azacitidine, 5-aza-2′-deoxycytidine, clofarabine, gaudecitabine, GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UVI5008.
43. The use of a compound of Formula I:

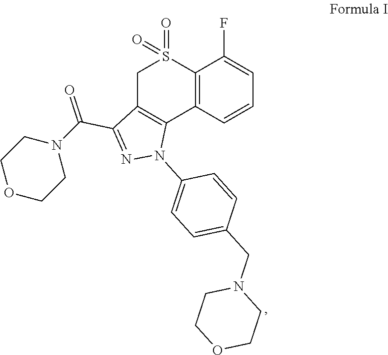 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, in the manufacture of a medicament for use in the treatment of a haematological malignancy, wherein said treatment comprises the separate, sequential or simultaneous administration of i) said medicament comprising Compound 1, or a pharmaceutically acceptable salt thereof, and ii) a HDAC inhibitor, or a pharmaceutically acceptable salt thereof, to said subject.
44. The use of Compound 1, or a pharmaceutically acceptable salt thereof, according to statement 43, wherein the haematological malignancy is lymphoma, leukaemia, myeloma, myelodysplastic syndrome or myeloproliferative diseases.
45. The use of Compound 1, or a pharmaceutically acceptable salt thereof, according to statement 44, wherein the lymphoma is cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.
46. The use of Compound 1, or a pharmaceutically acceptable salt thereof, according to statement 44, wherein the leukemia is ALL, AML, CLL, chronic myelogenous leukemia, hairy cell leukemia, myelodysplastic syndromes, myeloproliferative disorders and myelofibrosis.
47. The use of Compound 1, or a pharmaceutically acceptable salt thereof, according to any one of statements 43 to 46, wherein the HDAC inhibitor is selected from Abexinostat, Pracinostat, Guisinostat, Tefinostat, Panobinostat, Belinostat, Givinostat, Tucidinostat, Vorinostat, Mocetinostat, Valproic acid, Entinostat, Romidepsin, and Trapoxin.
48. The use of Compound 1, or a pharmaceutically acceptable salt thereof, according to any one of statements 43 to 47, wherein Compound 1 is administered as the hemifumarate salt in a dose of 40 mg or 80 mg per day.
49. The use of Compound 1, or a pharmaceutically acceptable salt thereof, according to any one of statements 43 to 48, wherein a HDAC inhibitor or a pharmaceutically acceptable salt thereof is administered in a dose between 10 mg and 1 g per day.
50. The use of Compound 1, or a pharmaceutically acceptable salt thereof, according to any one of statements 43 to 49, wherein the HDAC inhibitor is selected from Vorinostat or a pharmaceutically acceptable salt thereof.
51. The use of Compound 1, or a pharmaceutically acceptable salt thereof, according to any one of statements 43 to 50, wherein the HDAC inhibitor is Vorinostat or a pharmaceutically acceptable salt thereof and is administered at a dose of 200 mg, 300 mg or 400 mg per day.
52. The use of Compound 1, or a pharmaceutically acceptable salt thereof, according to any one of statements 43 to 51, wherein the HDAC inhibitor is Romidepsin.
53. The use of Compound 1, or a pharmaceutically acceptable salt thereof, for use according to any one of statements 43 to 52, wherein the treatment further comprises the separate, sequential or simultaneous administration of iii) a further chemotherapeutic agent, to said subject.
54. The use of Compound 1, or a pharmaceutically acceptable salt thereof, for use according to statement 53, wherein the further chemotherapeutic agent is a second HDAC inhibitor.
55. The use of Compound 1, or a pharmaceutically acceptable salt thereof, for use according to statement 53, wherein the further chemotherapeutic agent is a DNA methyltransferase inhibitor.
56. The use of Compound 1, or a pharmaceutically acceptable salt thereof, for use according to statement 55, wherein the DNA methyltransferase inhibitor is selected from 5-azacitidine, 5-aza-2′-deoxycytidine, clofarabine, gaudecitabine,GSK3685032 RX-3117, 5-fluoro-2-deoxycytidine, 6-dihydro-5-azacytidine, fazarabine, cladribine, fludarabine, procaine, epgallocatechin gallate, hydralazine, genistein, equol, curcumin, disulfiram, resveratrol, caffeic acid, CP-4200, zebularine, NPEOC-DAC, T-dCyd, 5-aza-t-dCyd, RG108, DC-05, DC-501, DC-517, SGI-1027, CM-272, CM-579, Psammaplin A, Psammaplin G and UVI5008.
57. A pharmaceutical product comprising i) a compound of Formula I:

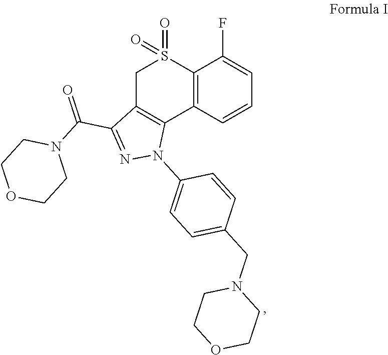 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof, and ii) a HDAC inhibitor or a pharmaceutically acceptable salt thereof.
58. A pharmaceutical product according to statement 57, wherein the pharmaceutical product further comprises iii) a further chemotherapeutic agent.
59. A kit comprising: a first pharmaceutical composition comprising a compound of Formula I:

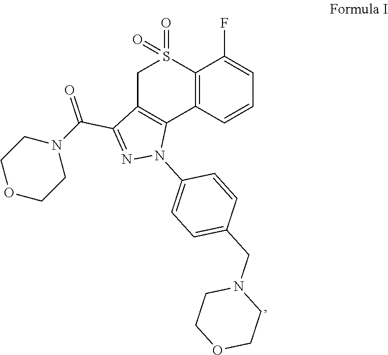 [SMILES: O=C(c1nn(-c2ccc(CN3CCOCC3)cc2)c2c1CS(=O)(=O)c1c(F)cccc1-2)N1CCOCC1]

or a pharmaceutically acceptable salt thereof; a second pharmaceutical composition comprising a HDAC inhibitor, or a pharmaceutically acceptable salt thereof; and instructions for using the first and second pharmaceutical compositions in combination.
60. A kit according to statement 59, wherein the kit further comprises a third pharmaceutical composition comprising a further chemotherapeutic agent, and the instructions are for using the first, second and third pharmaceutical compositions in combination.

1. A method of treating a haematological malignancy in a subject in need thereof, the method comprising administering to the subject a first amount of a compound of Formula I: 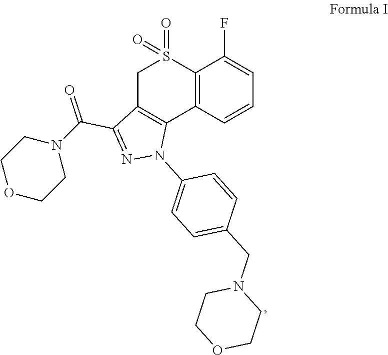 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof, and a second amount of a HDAC inhibitor, or a pharmaceutically acceptable salt thereof, wherein the first amount and the second amount together comprise a therapeutically effective amount.

2. The method according to claim 1, wherein the haematological malignancy is lymphoma, leukaemia, myeloma, myelodysplastic syndrome or myeloproliferative diseases.

3. The method according to claim 2, wherein the lymphoma is cutaneous B-cell lymphoma, cutaneous T-cell lymphoma, Hodgkin's lymphoma, Non-Hodgkin's lymphoma, FL, mantle cell lymphoma (MCL), MZL, DLBCL, SLL, PTCL and Waldenstrom macroglobulinemia.

4. The method according to claim 2, wherein the leukemia is ALL, AML, CLL, chronic myelogenous leukemia, hairy cell leukemia, myelodysplastic syndromes, myeloproliferative disorders and myelofibrosis.

5. The method according to claim 1, wherein the HDAC inhibitor is selected from Abexinostat, Pracinostat, Guisinostat, Tefinostat, Panobinostat, Belinostat, Givinostat, Tucidinostat, Vorinostat, Mocetinostat, Valproic acid, Entinostat, Romidepsin, and Trapoxin.

6. The method according to claim 1, wherein Compound 1 is administered as a hemifumarate salt in a dose of 40 mg or 80 mg per day.

7. The method according to claim 1, wherein the HDAC inhibitor or pharmaceutically acceptable salt thereof is administered in a dose between 10 mg and 1 g per day.

8. The method according to claim 1, wherein the HDAC inhibitor is selected from Vorinostat or a pharmaceutically acceptable salt thereof.

9. The method according to claim 1, wherein the HDAC inhibitor is Vorinostat or a pharmaceutically acceptable salt thereof and is administered at a dose of 200 mg, 300 mg or 400 mg per day.

10. The method according to claim 1, wherein the HDAC inhibitor is Romidepsin.

11. The method according to claim 1, wherein the treatment further comprises the separate, sequential, or simultaneous administration of a further chemotherapeutic agent, to said subject, wherein the further chemotherapeutic agent is a second HDAC inhibitor or a DNA methyltransferase inhibitor.

12-22. (canceled)

23. A pharmaceutical product comprising i) a compound of Formula I: 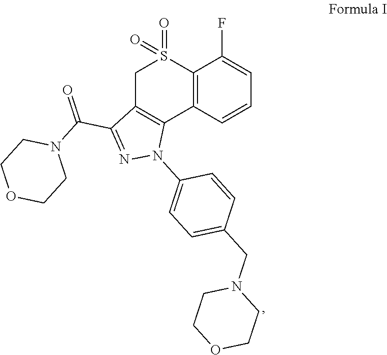 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof, and ii) a HDAC inhibitor or a pharmaceutically acceptable salt thereof.

24. A pharmaceutical product according to claim 23, wherein the pharmaceutical product further comprises iii) a further chemotherapeutic agent.

25. A kit comprising: a first pharmaceutical composition comprising a compound of Formula I: 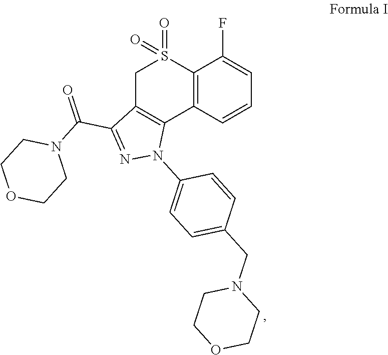 [SMILES: O=C(c1nn(-c2ccc(CN3CCOCC3)cc2)c2c1CS(=O)(=O)c1c(F)cccc1-2)N1CCOCC1]
or a pharmaceutically acceptable salt thereof; a second pharmaceutical composition comprising a HDAC inhibitor, or a pharmaceutically acceptable salt thereof; and instructions for using the first and second pharmaceutical compositions in combination.

26. The kit according to claim 25, wherein the kit further comprises a third pharmaceutical composition comprising a further chemotherapeutic agent, and the instructions are for using the first, second, and third pharmaceutical compositions in combination.
